(19) United States
(12) Patent Application Publication
(10) Pub. No.: US 2020/0253260 A1
(43) Pub. Date: Aug. 13, 2020

(54) Food Supplement For Protecting Female and Male Fertility

(71) Applicant: NURIBIOL, Lyon (FR)

(72) Inventor: Marc Cohen, Lyon (FR)

(21) Appl. No.: 16/630,325

(22) PCT Filed: Jun. 29, 2018
(86) PCT No.: PCT/FR2018/051623

§ 371 Date: Apr. 16, 2020

(30) Foreign Application Priority Data
Jul. 10, 2017 (FR) 1756514

(51) Int. Cl. A23L 33/15; A23L 33/16; A23L 33/175; A61P 15/08

(52) U.S. Cl. CPC A23L 33/15; A23L 33/16; A61K 9/20; A61P 15/08; A23L 33/175

(57) ABSTRACT

The food supplement according to the present invention for protecting female and male fertility intended for oral administration, helping reduce both oxygenated free radicals, which cause DNA damage, and homocystine, therefore reducing the deleterious action of same on methylation, comprises, in combination: A first ingredient consisting of zinc bisglycinate; A second ingredient formed from a natural amino acid from the N-acetyl-L cysteine amino acid family; A first vitamin (B12) belonging to the cyanocobalamin family in a methylated form; A second vitamin (B2) from the Riboflavin family; A third vitamin (B6) originating from the pyridoxine chlorhydrate family; And a third ingredient formed from a levomefolic acid and, more particularly, from 5-methyltetrahydrofolic acid.

The present invention relates to a food supplement used for protecting female and male fertility.

The causes of fertility disorders are found at four levels: sperm quality; ovulation; the female and male genital tracts; and incompatibility between sperm and female genitalia.

Also, hypofertility, or difficulties in conceiving, is certainly multifactorial but includes two major negative factors: oxidative stress and alterations in methylation of DNA of the gametes, then of the embryo.

Numerous studies have demonstrated the decline in sperm quality of fertile men over the last twenty years, which would cause an alteration of fertility in future years.

In the industrialized countries 15% of couples are infertile; the identified causes vary, but a large proportion show no anomaly at the level of each spouse, yet they fail to conceive a child; this is unexplained infertility.

Oxidative stress explanation: 98% of inhaled oxygen is reduced to water molecules by capture of four electrons and is accompanied by ATP formation, which is responsible for production of energy by the cell for its defense, mobility, and reproduction.

But in 2% of cases there is a leakage of electrons, which directly react with oxygen. This oxygen will be affected by a single electron, which will lead to formation of toxic reactive oxygen species.

Oxidative stress is the main cause of pathological spermograms because it causes DNA fragmentation and chromatin disruption. Oxidative stress in particular is responsible for shortening of telomeres and fragmentation of the sperm DNA.

This damage to the DNA, if not repaired in the new site at the time of fertilization, will harm the quality of the embryo or even the conceptus.

Oxidative stress (OS) factors may be endogenous or exogenous. The mitochondrion generates oxidative free radicals (ROS) via its sugar metabolism (OXPHOS), but oxidases (NADPH, xanthine, glycolate) also generate OS.

However, the exogenous sources of OS are also well known: pesticides, xenobiotics, cigarettes, herbicides, various drugs, etc. The role of oxidative stress on the alterations of spermatogenesis is clear.

It is the work of D. Evenson et al. (1980) that enabled an understanding of the processes. The OS induces fragmentation of the DNA in particular by formation of abasic (apuric or apyrimidic) sites.

Guanine is the most sensitive base for oxidation, and the monitoring of formation of its oxidation product, 8 OH dG, is fully correlated with DNA fragmentation. Telomeres with TTAGGG nucleotide repeats, which are very sensitive to oxidation, are shortened by oxidative stress in relation to the DNA fragmentation. However, there are more than 20 compounds that are oxidation products of nuclear bases.

In addition, OS produces additions, condensation products of the bases with lipid oxidation products or even vinyl. Fragmentation and other formations of base oxidation products are normally repaired in the ovocyte at the time of fertilization. However, this possibility of repair is finished and decreases with age.

Methylation is a biochemical process that modifies the gene expression, whose damage causes transgenerational pathologies severely affecting reproduction but also general metabolism, with the risk of diabetes and obesity but also inducing mental pathologies including autism.

Methylation is a key biochemical process that affixes covalent methyl groups on lipids, proteins and DNA. In the case of epigenetics this is of course DNA and histones related thereto; the universal cofactor is S-adenosylmethionine, which after methylation of the target protein forms the S-adenosylhomocysteine, then homocysteine.

This molecule must be recycled to methionine, via the methionine (1-CC) cycle. Homocysteine is a methylation inhibitor. It is classed as a cause and consequence of oxidative stress.

It can be said that it is responsible for OS and methylation problems. The methylation of DNA occurs at the level of CPG “islands,” repetitive sequences of CpG, associated with or very close to the promoters, control zone and/or regulation of the genes.

It modifies the structure of the chromatin and thus the functionalities of the genome. These changes are heritable and likely to change according to exogenous parameters related to the environment, food, pollution, or drugs.

Thus, oxidative stress and methylation are two sides of the same coin, having in common the possibility of repairing the methionine cycle.

Oxidative stress results from the action of age, endocrine disruptors of chronic inflammation, and nutritional deficiency (especially of group B).

The object of the present invention is to design a food supplement whose composition allows reduction of oxidative stress and protection of methylation.

Oxidative stress results in formation of oxygen free radicals responsible for cellular damage both directly and through a harmful effect on methylation.

The composition of the food supplement according to the present invention allows endogenous production of glutathione, a universal protector against oxidative stress, in parallel with the recycling of homocysteine in the methionine cycle, which has the effect of reducing the circulating homocysteine, thereby reducing its harmful effect on methylation.

The most effective way to reduce their harmful action is to promote endogenous production of glutathione: this is the first pillar of defense.

For this we must introduce a series of amino acids that are the precursors of glutathione: these are glycine, glutamic acid, cysteine, cystine and N-acetylcysteine, to which is added vitamin B6 that stimulates their endogenous production.

The second pillar of defense concerns the methionine cycle aimed at producing CH3 methylation stabilizers and at the same time reducing the circulating homocysteine, thus reducing its harmful action on methylation.

For this a group of vitamins was selected from vitamins B2, B12 and B9 associated with zinc bisglycinate and betaine, ensuring an increase in methionine in order to yield an amino acid of the family S-adenosylmethionine (SAM), which releases stabilizers of CH3 methylation causing a reduction of circulating homocysteine through S-adenosylhomocysteine (SAH).

In addition, in place of conventional folic acid, which does not play its beneficial role in the methionine cycle, in case of mutation of the enzyme methyltetrahydrofolate reductase (MTHFR), in this new composition the preference has been to take directly reduced folic acid, namely 5-methyltetrahydrofolate (SMTHF).

As part of this second pillar of cellular defense resulting in stabilization of methylation and in parallel the reduction of circulating homocysteine by vitamins B2, B12 associated with folic acid 5-methyltetrahydrofolate (SMTHF), zinc bisglycinate, betaine, and amino acid of the family S-adenosylmethionine (SAM) play a decisive role.

The object of the present invention is a food supplement used to protect female and male fertility.

The food supplement according to the present invention for protecting female and male fertility making it possible to reduce on the one hand the oxygen free radicals causing DNA damage and, on the other hand, the action on homocysteine in its harmful action on methylation, comprises in combination:
a first ingredient consisting of zinc bisglycinate;
a second ingredient formed from a naturally occurring amino acid of the amino acid family N-acetyl-L cysteine;
a first vitamin (B12) belonging to the cyanocobalamin family in a methylated form;
a second vitamin (B2) from the riboflavin family;
a third vitamin (B6) from the pyridoxine hydrochloride family; and
a third ingredient formed from a levomefolic acid, and more particularly from 5-methyltetrahydrofolic acid

The food supplement according to the present invention may comprise an amino acid reinforcement comprising in combination cystine, glycine and glutamic acid.

The food supplement according to the present invention may comprise in combination betaine and another amino acid of the S-adenosylmethionine family.

The food supplement according to the present invention may comprise in combination components previously described, vitamin B3, or niacin.

Preferably, the vitamin B3 is present in the food supplement of the invention with a concentration between 2.4 and 80 mg, more preferably between 2.4 and 32 mg.

The food supplement according to the present invention may comprise in combination components previously described, NADH.

NADH, or nicotinamide adenine dinucleotide in reduced form, is involved in metabolism as an electron carrier in redox reactions, NAD+ as oxidant and NADH as reducing agent.

Preferably, the NADH is present in the food supplement of the invention with a concentration between 5 and 100 mg, more preferably between 5 and 50 mg.

The food supplement according to the present invention may comprise in combination components previously described, myo-inositol or D-chiro-inositol (two different forms of the inositol molecule).

Myo-inositol was formerly improperly called vitamin B7, even though it is not a vitamin because it is synthesized in the human body.

Myo-inositol plays an important role as a secondary messenger in eukaryotic cells, especially in the form of inositol phosphate, phosphatidylinositol (PI), and phosphatidylinositol phosphate (PIP).

The inositol molecule has multiple effects on our health. For example, it makes it possible to increase resistance to oxidative stress. However, it is mainly used to fight type 2 diabetes. This diabetes is characterized by insulin resistance, causing an increase in blood sugar levels. Inositol helps to improve insulin sensitivity in people suffering from insulin resistance.

Insulin resistance seems to be related to polycystic ovary syndrome (PCOS), the most common reason for female infertility: 5 to 10% of women are affected by it. This syndrome is characterized by an increase of androgens, the male hormones linked to insulin resistance. An excessively high level of androgens (hyperandrogenism) is often the cause of a disturbance of the menstrual cycle in addition to a lack of sensitivity to insulin. A disruption of the menstrual cycle causes a drop or even a total cessation of ovulation.

Preferably, myo-inositol or D-chiro-inositol is present in the food supplement of the invention with a concentration between 1 and 5 g, more preferably between 2 and 4 g, more preferably between 2 and 4 mg.

The food supplement according to the invention preferably matches the composition for a tablet or capsule:
at least 15 mg of zinc,
45 to 65 mg of amino acids N-acetyl-L cysteine,
2 to 3 μg of vitamin B12 from the cyanocobalamin family,
1 to 2 mg of vitamin B2 from the riboflavin family,
1 to 2 mg of vitamin B6 from the pyridoxine hydrochloride family,
450 to 580 μg of vitamin B9 from 5-methyltetrahydrofolic acid

The food supplement according to the present invention can comprise in a tablet or capsule:
at least 15 mg of zinc,
45 to 65 mg of amino acids N-acetyl-L cysteine,
2 to 3 μg of vitamin B12 from the cyanocobalamin family,
1 to 2 mg of vitamin B2 from the riboflavin family,
1 to 2 mg of vitamin B6 from the pyridoxine hydrochloride family,
450 to 580 μg of vitamin B9 from 5-methyltetrahydrofolic acid,
30 to 40 mg of cystine,
30 to 40 mg of glutamic acid,
30 to 40 mg of glycine

The food supplement according to the present invention can comprise in a tablet or capsule:
at least 15 mg of zinc,
45 to 65 mg of amino acids N-acetyl-L cysteine,
2 to 3 μg of vitamin B12 from the cyanocobalamin family,
1 to 2 mg of vitamin B2 from the riboflavin family,
1 to 2 mg of vitamin B6 from the pyridoxine hydrochloride family,
450 to 580 μg of vitamin B9 from 5-methyltetrahydrofolic acid,
30 to 40 mg of cystine,
30 to 40 mg of glutamic acid,
30 to 40 mg of glycine,
35 to 45 mg of betaine,
30 to 40 mg of S-adenosylmethionine.

The food supplement according to the present invention can comprise in a tablet or capsule:

at least 15 mg of zinc,
45 to 65 mg of amino acids N-acetyl-L cysteine,
2 to 3 μg of vitamin B12 from the cyanocobalamin family,
1 to 2 mg of vitamin B2 from the riboflavin family,
1 to 2 mg of vitamin B6 from the pyridoxine hydrochloride family,
450 to 580 μg of vitamin B9 from 5-methyltetrahydrofolic acid,
30 to 40 mg of cystine,
30 to 40 mg of glutamic acid,
30 to 40 mg of glycine,
35 to 45 mg of betaine,
2 to 32 mg of B3,
5 to 50 mg of NADH,
2 to 4 gr of myo-inositol.

The various food supplement compounds can be administered simultaneously (in the same tablet, for example) or as combination products for separate use or spread over time. They are preferably administered simultaneously.

The food supplement is formulated to be administered orally. The food supplement according to the invention may also be formulated to be administered transrectally or to be injected.

The food supplement according to the invention can be formulated as a solid or liquid composition. It can thus be in the form of a capsule, pill, tablet, granule, suppository, and more preferably in the form of a tablet.

The liquid form can be selected from among soluble granules, an oral solution, or injectable solutions.

When the food supplement is in the form of a soft capsule or pill, the envelope of these soft capsules or these pills may contain in particular animal gelatin such as fish gelatin, glycerin, or a material of plant origin such as a cellulose or starch derivative or a vegetable protein.

When the food supplement is in capsule, tablet, or granule form, the active mixture can be attached to a powdery carrier.

Advantageously according to the present invention, the food supplement may comprise any suitable vehicle or excipient acceptable from a nutraceutical point of view, as well as conventional additives known to the person skilled in the art.

Advantageously according to the present invention, the food supplement of the present invention makes it possible to reduce circulating homocysteine in the blood of subjects who carry the standard genotypes of MTHFR and MTRR and of subjects who carry other pathogenic isoforms of said genes such as the mutations C677T and/or A1298C.

According to another aspect, another object of the present invention is use of the aforementioned food supplement as a perinatal supplement and/or pregnancy supplement.

In a preferred embodiment, the supplement according to the invention can be used as a food supplement for men and women to promote conception, at least for 12 months, preferably for at least 46 months, preferably for at least 3 months before conception.

In women, premature ovarian failure (POF) and diminished ovarian reserve (DOR) syndromes can be produced by environmental factors causing methylation and epigenetic errors.

The probability of conceiving is directly related to the store of ovarian follicles known as “ovarian reserve.” A decrease in this stock of follicles and ovocytes is accompanied by a lower pregnancy rate and a higher miscarriage rate.

The drop in the ovarian reserve is the main cause of decreased fertility with age.

FSH secreted by the pituitary gland (associated with LH) stimulates follicle development, the production of estradiol by the follicle during the follicular phase of a menstrual cycle. It works in synergy with LH to cause ovulation.

The concentration of estradiol increases slowly and regularly at the beginning of the follicular phase (recruitment), then much more rapidly when the dominant follicle is selected (second part of the follicular phase).

The elevation of estradiol (E2) in parallel with this causes a fall in FSH, by negative feedback, as by the way INHB also does, whose production is also under the influence of FSH.

The anti-Müllerian hormone (AMH) reportedly inhibits the growth of small follicles and thus participates in development of the dominant follicle. The ovarian secretion of AMH increases gradually at puberty while remaining at very low levels; it decreases with age to become undetectable at menopause.

The depletion of follicles (decrease in ovarian reserve) will lower the rate of circulating INHB responsible for an elevation of FSH, itself responsible for an elevation of estradiolemia.

The AMH rate decreases well before FSH increases, which for some authors would make it an early marker of ovarian depletion.

As demonstrated by Example 3, the supplement of the invention allows a significant increase in AMH levels and thus surprisingly improves ovarian function.

According to another aspect, another object of the present invention is use of the food supplement mentioned previously to treat symptoms of premature ovarian failure (POF) and diminished ovarian reserve (DOR); i.e. to restore the metabolic balance and reestablish ovulation and fertility.

According to another aspect, another object of the present invention is use of the food supplement mentioned previously for treatment of polycystic ovary syndrome (PCOS).

In a particular embodiment, the food supplement according to the present invention for treating POF and DOR comprises:
at least 15 mg of zinc,
45 to 65 mg of amino acids N-acetyl-L cysteine,
2 to 3 μg of vitamin B12 from the cyanocobalamin family,
1 to 2 mg of vitamin B2 from the riboflavin family,
1 to 2 mg of vitamin B6 from the pyridoxine hydrochloride family,
450 to 580 μg of vitamin B5 from 5-methyltetrahydrofolic acid,
30 to 40 mg of cystine,
30 to 40 mg of glutamic acid,
30 to 40 mg of glycine,
35 to 45 mg of betaine,
2 to 32 mg of B3,
5 to 50 mg of NADH,
2 to 4 gr of myo-inositol.

In a particular embodiment, the food supplement according to the present invention for treating polycystic ovary syndrome (PCOS) comprises:
at least 15 mg of zinc,
45 to 65 mg of amino acids N-acetyl-L cysteine,
2 to 3 μg of vitamin B12 from the cyanocobalamin family,
1 to 2 mg of vitamin B2 from the riboflavin family,
1 to 2 mg of vitamin B6 from the pyridoxine hydrochloride family,
450 to 580 μg of vitamin B9 from 5-methyltetrahydrofolic acid,
30 to 40 mg of cystine,
30 to 40 mg of glutamic acid,
30 to 40 mg of glycine,
35 to 45 mg of betaine,
2 to 32 mg of B3,
5 to 50 mg of NADH,
2 to 4 gr of myo-inositol.

As explained before, several disorders or problems of female and male fertility, such as sperm DNA fragmentation (% DFI) and sperm DNA decondensation (% SDI), cause oxidative stress. The component of the invention allows reduction of the oxygen free radicals causing damage to the DNA and the effect on homocysteine in its harmful effect on methylation.

The invention also has the object of using a supplement such as defined above to produce a medicament for oral administration to treat: problems of female and male fertility such as miscarriage; premature ovarian insufficiency; abnormal sperm parameters; premature ovarian failure (POF) syndromes; diminished ovarian reserve (DOR), polycystic ovarian syndrome (PCOS); and/or endometriosis.

Another object of the invention is a supplement according to the invention (or supplement of the invention) intended for use in treating female and male fertility problems such as miscarriage, premature ovarian insufficiency, abnormal sperm parameters, premature ovarian failure (POF) syndromes, diminished ovarian reserve (DOR), polycystic ovary syndrome (PCOS), and/or endometriosis, characterized in that it involves oral administration of a food supplement as defined above.

Another object of the invention is a dietary treatment method, characterized in that it involves oral administration of one of the food supplements according to the invention.

Another object of the invention is a dietary food supplement according to the invention for treating problems of female and male fertility such as miscarriage, premature ovarian insufficiency, abnormal sperm parameters, premature ovarian failure (POF) syndromes, diminished ovarian reserve (DOR), polycystic ovary syndrome (PCOS), and/or endometriosis.

### EXAMPLES

### Example 1

Experiment no. 1 shows a clinical study of 30 couples with fertility problems for 4 years such as miscarriage, premature ovarian insufficiency, or abnormal sperm parameters and with two-thirds having already experienced IVF failures.

At least one person of the pair has been tested for the isoform C677T of methylenetetrahydrofolate reductase (MTHFR). The couples were treated with food supplement 1 (FIG. 1) for 4 months before starting a new IVF.

FIG. 1: List of ingredients of food supplement 1.

| List of ingredients | mg/capsule | Intake for 1 capsule | % VNR |
|---|---|---|---|
| zinc bisglycinate | 79 | 15 mg of zinc | 150 |
| N-acetyl L-cysteine | 50 | | |
| betaine hydrochloride | 40 | | |
| L-cystine | 35 | | |
| L-glutamine | 35 | | |
| L-glycine | 55 | | |
| S-adenosylmethionine | 35 | | |
| cyanocobalamin | 2.5 | 2.5 mg vitamin B12 | 100 |
| (0.1% vitamin B12) | | | |
| pyridoxine hydrochloride | 1.68 | 1.4 mg vitamin B6 | 100 |
| riboflavin | 1.4 | 1.4 mg vitamin B2 | 100 |
| methyltetrahydrofolic acid | 0.92 | 0.5 mg vitamin B9 | 250 |
| glucosamine salt | | | |
| magnesium stearate | 11.25 | | |
| silica | 32.23 | | |
| capsule | 75 | | |
| weight | 434 | | |

For the mutation C677T the distribution of women is: patients without mutations (WT): 38%, heterozygotes (HTZ): 45%, homozygotes (HMZ): 17%. For the men, 6 patients were HMZ.

The result of taking the invention's food supplement 1 is 13 spontaneous pregnancies; 13 pregnancies after IVF treatment (42%) and only 10% (3 couples) without pregnancy. Treatment with the invention's food supplement 1 allows a surprising improvement of female and male fertility even in the case of patients with mutations in the MTHFR gene.

### Example 2

Experiment no. 2 shows a clinical study in 60 couples with an infertility problem for at least 4 years such as miscarriage and premature ovarian insufficiency.

The women were tested for the isoform C677T (mutation of the MTHFR gene). If the woman was positive the man was also tested. All carriers of the mutation were tested for homocysteine in the blood before starting the three-month treatment with the food supplement 1 described in Example 1.

Results:

As expected, the circulating homocysteine was higher in the homozygote (HMZ) patients: 18.4 micromoles/L, than in the heterozygote (HTZ) patients: 12.2 micromoles/L. The patients without mutations (WT) had a concentration of 7.8 micromoles/L.

The treatment causes an overall reduction of 32.6% (from 14.9 to 10.1 micromoles/L) in people with the mutation.

The treatment with food supplement 1 is effective even in people with a mutation in the MTHFR gene and reduces the circulating homocysteine so as to promote an improvement in gametogenesis and probability of pregnancy.

### Example 3

In women, syndromes of premature ovarian failure (POF) and diminished ovarian reserve (DOR) can be caused by environmental factors that produce methylation and epigenetic errors.

Example 3 shows two clinical studies. A first study with a group (A) of 20 patients with a clinical history of infertility for 3 to 7 years and with at least 2 IVF failures. This group was tested for the anti-Müllerian hormone (AMH) before and after the 4-month treatment with food supplement 1.

A second group of patients (35, group B) had low chronic serum levels of AMR (<1 ng/ml). The levels of bisphenol A (BPA) are measured before starting treatment. In group B, the treatment with supplement 1 significantly raises the AMR levels independent of the BPA concentration in the urine.

In all patients (A+B=49) there is a significant increase in AMH levels (p=0.0001) after the treatment irrespective of the initial levels of AMR and the age of the woman (see FIG. 2); five patients had a spontaneous pregnancy 2 months after the end of the treatment.

The AMH levels in the blood have a direct correlation with the functional reserve of the ovary and are considered a biomarker of ovarian function.

This experiment demonstrates that the invention's supplement 1 improves ovarian function.

### Example 4

We carried out a clinical study in 69 men who had already experienced two IVF failures and were waiting for a third attempt. Treatment with supplement 1 for couples with male problems yielded very surprising results. The analysis of sperm DNA fragmentation (% DFI) is measured with the SDF test, and the analysis of sperm DNA decondensation (% DSI) is measured with the SDI test (sperm DNA integrity test) (Hamidi J. et al. Zygote, 2015; 23:556-62).

FIG. 2: SDF pre- and post-treatment.

| | | Parents | SDF | | |
|---|---|---|---|---|---|
| | N | age (yy) | Pre-treatment | Post-treatment | Δ (p*) |
| Treatment with | 69 | 378 | 24.6 | 20 | −19% |
| supplement 1 | | (6.9) | (12.4) | (12.7) | (0.000) |
| (p**) | | | | | <0.01 |
| Controls | 84 | 37.8 | 30.9 | 31.2 | +1% |
| | | (4.5) | (34.4) | (34.3) | (0.322) |

Average values (+/−deviation type:
Δ = observed differences between the groups pre- and post-treatment:
p* = p value vs. pre Rx (Wilcoxon test):
p** = p value vs. controls (Mann Whitney test)

FIG. 3: SDI pre- and post-treatment.

| | | Parents | SDF | | |
|---|---|---|---|---|---|
| | N | age (yy) | Pre-treatment | Post-treatment | Δ (p) |
| Treatment with | 59 | 37.6 | 41.7 | 35.0 | −16% |
| supplement 1 | | (5.9) | (9.7) | (15.0) | (0.001) |
| (p**) | | | | | <0.01 |
| Controls | 84 | 37.8 | 30.5 | 31.1 | +2% |
| | | (4.5) | (6.4) | (3.5) | (0.148) |

Average values (+/−deviation type:
Δ = observed differences between the groups pre- and post-treatment:
p* = p value vs. pre Rx (Wilcoxon test):
p** = p value vs. controls (Mann Whitney test)

In the men, the sperm DNA fragmentation and sperm DNA decondensation rates decline very strongly after treatment (p<0.001).

### Example 5

Endometriosis

The presence of the MTHFR mutation was investigated by standard methods among 30 non-fertile patients suffering from endometriosis and who had tried at least one unsuccessful IVF. 60% of the patients carried the MTHFR mutation (46.7% heterozygotes, 13.3% homozygotes).

This proportion is significantly higher than the frequency of the mutation in the general population (50.5%; Zappacosta et al. 2014)

The patients carrying the MTHFR mutation were then treated with the food supplement of the invention no. 1.

The pregnancy rate was compared before and after treatment. The successful IVF rate was significantly increased (mean pregnancy rate per cycle: 23.4% before treatment, 29.6% after treatment).

### Example 6

Experiment no. 6 shows a clinical study of couples with an infertility problem for at least 4 years such as miscarriage, premature ovarian insufficiency, and polycystic ovary syndrome (PCOS).

All the couples (men and women) are tested for the presence or absence of mutation of the gene encoding MTHFR: two types of mutation (C677T and A1298C).

The men are tested before and after treatment with one capsule per day of food supplement 2 for: exploration of the spermogram; analysis of sperm DNA fragmentation (% DFI); analysis of sperm DNA decondensation (% DSI); and exploration of an oxidative stress marker: homocysteine level.

One group is treated with a placebo (common folic acid).

The women are tested before and after the treatment with one capsule per day of food supplement 2 for: exploration of ovarian insufficiency (measurement of AMH rate. AMH <1.5 μg/mL) and exploration of an oxidative stress marker: homocysteine level.

One group is treated with a placebo (common folic acid).

The patients (men and women) will take the treatment for 4 months. For the woman benefiting, in case of pregnancy she will continue to take the treatment until the 12th week of amenorrhea.

This test is designed to show it is possible to obtain a reduction in the homocysteine level in the event of a mutation of the MTHFR gene by giving the patient a food supplement of the invention no. 2. This is intended to increase the chances of pregnancy.

FIG. 4: Ingredients of the food supplement no. 2

| Ingredients (active) | mg/unit |
|---|---|
| ZINC BISGLYCINATE 20% | 75 |
| N ACETYL CYSTEINE | 50 |
| VITAMIN B3 (PP) 97.9% | 39.22 |
| CYSTINE | 35 |
| GLUTAMIC ACID | 35 |
| Glycine | 35 |
| Methionine L | 35 |
| INOSITOL* | 2500 |
| VITAMIN B12 0.1% | 3 |
| VITAMIN B6 80.46% | 2.088 |
| VITAMIN B2 97.5% | 1.72 |
| VITAMIN B9 87.5% | 0.6857 |
| NADH | 6.67 |
| Ingredients (excipients) | mg/unit |
| MALTODEXTRIN | 1000 |
| ANHYDRIC CITRIC ACID | 50 |
| STEVIA REBAUDIOSIDE EXTRACT DRY 97% | 4 |
| Peche arA'me | 400 |
| TIXOSIL 38 SILICA PRECIPITATED | 2 |

1. A food supplement to protect male and female fertility for oral administration, thereby reducing both the oxygen free radicals causing DNA damage and the action on homocysteine in its harmful effect on methylation, comprising in combination:
a first ingredient consisting of zinc bisglycinate;
a second ingredient formed from a naturally occurring amino acid of the amino acid family N-acetyl-L cysteine;
a first vitamin (B12) belonging to the cyanocobalamin family in a methylated form;
a second vitamin (B2) from the riboflavin family;
a third vitamin (B6) from the pyridoxine hydrochloride family; and
a third ingredient formed from a directly reduced folic acid, and more particularly from 5-methyltetrahydrofolic acid.

2. The food supplement according to claim 1, characterized in that it comprises an amino acid reinforcement comprising in combination cystine, glycine, and glutamic acid.

3. The food supplement according to claim 1, characterized in that it comprises in combination betaine and another amino acid of the S-adenosylmethionine family.

4. The food supplement according to claim 1, characterized in that it comprises in a tablet or capsule:
at least 15 mg of zinc;
45 to 65 mg of amino acids N-acetyl L cysteine;
2 to 3 μg of vitamin B12 from the cyanocobalamin family;
1 to 2 mg of vitamin B2 from the riboflavin family,
1 to 2 mg of vitamin B6 from the pyridoxine hydrochloride family; and
450 to 580 μg of vitamin B9 from 5-methyltetrahydrofolic acid.

5. The food supplement according to claim 1, characterized in that it comprises in a tablet or capsule:
at least 15 mg of zinc;
45 to 65 mg of amino acids N-acetyl L cysteine;
2 to 3 μg of vitamin B12 from the cyanocobalamin family;
1 to 2 mg of vitamin B2 from the riboflavin family;
1 to 2 mg of vitamin B6 from the pyridoxine hydrochloride family;
450 to 580 μg of vitamin B9 from 5-methyltetrahydrofolic acid;
30 to 40 mg of cysteine;
30 to 40 mg of glutamic acid; and
30 to 40 mg of glycine.

6. The food supplement according to claim 1, characterized in that it comprises in a tablet or capsule:
at least 15 mg of zinc,
45 to 65 mg of amino acids N-acetyl L cysteine;
2 to 3 μg of vitamin B12 from the cyanocobalamin family;
1 to 2 mg of vitamin B2 from the riboflavin family;
1 to 2 mg of vitamin B6 from the pyridoxine hydrochloride family;
450 to 580 μg of vitamin B9 from 5-methyltetrahydrofolic acid;
30 to 40 mg of cysteine;
30 to 40 mg of glutamic acid;
30 to 40 mg of glycine;
35 to 45 mg of betaine; and
30 to 40 mg of S-adenosylmethionine.

7. The food supplement according to claim 1, characterized in that in addition it comprises vitamin B3 with a concentration between 2.4 and 80 mg, more preferably between 2.4 and 32 mg; and/or NADH with a concentration 5 and 100 mg, more preferably between 5 and 50 mg; and/or myo-inositol with a concentration 1 and 5 g, more preferably between 2 and 4 g, more preferably between 2 and 4 mg.

8. The food supplement according to claim 1, characterized in that it is realized in the form of a capsule, tablet, or granule, the active mixture is fixed on a powdery carrier.

9. The food supplement according to claim 1, characterized in that it is used as a perinatal supplement and/or a pregnancy supplement.

10. The food supplement according to claim 1, characterized in that it is used to reduce circulating homocysteine in the blood of subjects who carry the standard genotypes of MTHFR and subjects who carry other pathogenic isoforms of said gene like the C677T and/or A1298C mutations.

11. The food supplement according to claim 1, characterized in that it is used for treatment of female and male fertility problems such as miscarriage, premature ovarian insufficiency, abnormal sperm parameters, premature ovary failure (POF) syndromes, diminished ovarian reserve (DOR), polycystic ovary syndrome (PCOS), and/or endometriosis.
